(19) United States
(12) Patent Application Publication
(10) Pub. No.: US 2018/0155666 A1
(43) Pub. Date: Jun. 7, 2018

(54) Low Surface Energy Coatings For Mammalian Cell Culture

(71) Applicant: Trustees of Tufts College, Medford, MA (US)

(72) Inventors: Daniel J. Wilson, Mapleville, RI (US); Irene Lui, Golden, CO (US); Charles R. Mace, Medford, MA (US)

(21) Appl. No.: 15/832,287

(22) Filed: Dec. 5, 2017

Related U.S. Application Data
(60) Provisional application No. 62/430,765, filed on Dec. 6, 2016.

(51) Int. Cl. C12M 1/00; A01N 43/66; C12M 1/32; C12N 5/00; C12N 5/07; C12M 1/12

(52) U.S. Cl. CPC C12M 23/20; A01N 43/66; C12M 23/12; C12N 5/0068; C12N 2533/14; C12N 5/06; C12M 25/14; C12N 2533/30; C12N 5/0018

(57) ABSTRACT

A cultureware system is directed to maintaining viability of mammalian cells, and includes a disposable holding container having a cell receiver with a cell-receiving surface. The cell receiver consists of a cell-adhesion inducement material including a polystyrene material and a glass material. The system further includes a polytetrafluoroethylene (PTFE) coating lining the cell-adhesion inducement material of the cell-receiving surface, and a culture of adherent mammalian cells located within the cell receiver on the PTFE coating. In response to attachment interaction between the mammalian cells and the PTFE coating, a decreased cell adhesion results in a cell viability rate of at least about 60% to about 70% over a 72-hour culturing period, the cell viability rate being under 90% over the 72-hour culturing period if, in the absence of the PTFE coating, the mammalian cells are located directly on the cell-adhesion inducement material.

### CROSS-REFERENCE TO RELATED APPLICATIONS

This application claims priority to and the benefit of U.S. Provisional Patent Application Ser. No. 62/430,765, filed Dec. 6, 2016, and titled “Low Surface Energy Coatings For Mammalian Cell Culture,” which is incorporated herein by reference in its entirety.

### FIELD OF THE INVENTION

The present invention relates generally to cultureware, and, more particularly, to maintain viability of mammalian cells in a cell-culture holding container.

### BACKGROUND OF THE INVENTION

In general, culture of adherent mammalian cells requires a number of reagents and procedural steps to detach cells from disposable cultureware and to dispense them into additional containers for culture or analysis. This process is time intensive (i.e., it may require hours of work), with time often factoring heavy (and detrimentally) into the observations made and reported by researchers. Other problems associated with culturing of adherent mammalian cells are that required regents can have a detrimental effect on the cells being studied, cells are often lost or damaged during a transfer process, and cells continue to “change” (i.e., continue their life cycle) during the transfer process.

More specifically, the routine culture of anchorage-dependent cells typically relies on the use of disposable polystyrene or glass-bottom cultureware (e.g., dishes, flasks, or multi-well plates). Depending on the lineage and type, cells adhere to the surface of their container within minutes to hours of seeding. To conduct downstream experiments and analyses, however, cells must first be removed from these surfaces. Common dissociation techniques include enzymatic digestion of cell surfaces (e.g., using trypsin or Accutase) and the use of chelating reagents (e.g., EDTA and EGTA) to disrupt cell-cell/cell-surface interactions that are mediated by divalent cations. Mechanical methods (e.g., scraping) can also be used to remove cells from surfaces, but are harsh and less efficient than options that rely on reagents. Enzymatic approaches are unfavorable for studies requiring intact cell surfaces, as dissociation by trypsin may reduce the function of cell surface receptors, damage adhesion molecules, and cleave post-translational modifications.

For many applications, the proteolysis of cell surface proteins is detrimental because it results in a heterogeneous population of cells that is structurally and functionally different than cultured, adherent cells. Functional assays necessitate a substantial period of time (e.g., hours) to allow cells to recover from the changes in surface chemistry caused by these reagents before they are used in experiments. Furthermore, prolonged incubation in solutions of trypsin or other enzymatic cell dissociation reagents may cause membrane degradation, which can lead to a decrease in cell viability.

Non-enzymatic dissociation reagents (e.g., CellStripper and Versene) are composed of chelating agents that remove divalent cations (e.g., Ca²⁺ and Mg²⁺) from cell adhesion proteins that require these metals to function (e.g., E-cadherin, integrins, selectins). The resulting inhibition of these adhesion molecules allows for the removal of cells from cultureware. Although these reagents are significantly less destructive than their enzymatic counterparts, they require longer incubations (e.g., 5 minutes for trypsin vs. 30-45 minutes for EDTA) and mechanical shearing (e.g., mixing by pipette) to break cell-cell junctions. The reagents currently utilized are either time-intensive or may jeopardize the validity of experimental results.

Additionally, mammalian cell cultures typically require a minimum of 24 hours to double in number, making it necessary to wait 2-3 days after passaging before a suitable number of cells is reached for experimentation. A more time-efficient cell culture method, in which cells could be “held” in a container without adhering, would be beneficial as it would provide access to cells available for immediate use, without requiring the use of dissociation reagents or waiting for a lengthy growth period.

Polytetrafluoroethylene (“PTFE”), commercially known as TEFLON®, has been used previously in various cell-based experiments based on its non-stick properties. For suspension cultures, TEFLON® bags have been used for the collection and activation of killer T-cells, and to prevent differentiation of mononuclear phagocytes. TEFLON® surfaces have also been utilized in the culture of macrophages, primary cell lines, and stem cells.

Thus, previous use of TEFLON® bags and surfaces has failed to address the prevention of adhesion to a surface while maintaining the viability and functionality of the cells. There is a substantial need for a system and method that allows for easy, quick dispensing of intact and viable cells for experimentation.

### SUMMARY OF THE INVENTION

According to one aspect of the present invention, a cultureware system is directed to maintaining viability of mammalian cells, and includes a disposable holding container having a cell receiver with a cell-receiving surface. The cell receiver consists of a cell-adhesion inducement material including at least one of a plastic (e.g., polystyrene) material and a glass material. The system further includes a polytetrafluoroethylene (PTFE) coating lining the cell-adhesion inducement material of the cell-receiving surface, and a culture of adherent mammalian cells located within the cell receiver on the PTFE coating. In response to attachment interaction between the mammalian cells and the PTFE coating, a decreased cell adhesion results in a cell viability rate in the range of at least about 60% to about 70% over at least a 72-hour culturing period, the cell viability rate being under 90% over the at least 72-hour culturing period if, in the absence of the PTFE coating, the mammalian cells are located directly on the cell-adhesion inducement material.

According to another aspect of the present invention, a cell-culture holding container for cultured mammalian cells includes a receiver plate with a plurality of receiver wells, and a plurality of receiver inserts positioned, respectively, in the plurality of receiver wells. Each receiver insert of the plurality of receiver inserts has an internal receiver surface. The container further includes a polytetrafluoroethylene (PTFE) coating applied to each internal receiver surface, and a culture of adherent mammalian cells located within the plurality of receiver inserts . The PTFE coating is interposed between the mammalian cells and the respective internal receiver surface to impede cell adhesion to the internal receiver surface.

According to yet another aspect of the present invention, a method is directed to culturing mammalian cells in a holding container. The method includes providing a receiver plate with a plurality of receiver wells, and inserting a plurality of receiver inserts into respective ones of the plurality of receiver wells. The method further includes coating an internal receiver surface of each of the plurality of receiver wells with a polytetrafluoroethylene (PTFE) material to achieve a PTFE-coated receiver surface. The method also includes culturing adherent mammalian cells on the PTFE-coated receiver surface, and, in response to the culturing of the mammalian cells on the PTFE-coated receiver surface, achieving a cell viability rate of at least 70% over at least a 72-hour culturing period.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1A is an image of an individual well coated with a spray polytetrafluoroethylene (PTFE) material.

FIG. 1B is an image of an individual well coated with a modified PTFE material.

FIG. 1C is an image of an individual well coated with a virgin PTFE material.

FIG. 2 is a table showing recovery results of MDA-MB-231 cells from PTFE-coated wells.

FIG. 3 is a table with results showing recovery of HeLa and 3T3 cells from PTFE-coated wells over a period of 120 hours.

FIG. 4A is a chart with results showing cell viability of MDA-MB-231 cells.

FIG. 4B is a chart with results showing cell viability of HeLa cells.

FIG. 4C is a chart with results showing cell viability of 3T3 cells.

FIG. 5A is a chart with results of a cell cycle analysis by propidium iodide of MDA-MB-231 cells in wells coated with a spray PTFE material.

FIG. 5B is a chart with results of a cell cycle analysis by propidium iodide of MDA-MB-231 cells in wells coated with a modified PTFE material.

FIG. 5C is a chart with results of a cell cycle analysis by propidium iodide of MDA-MB-231 cells in wells coated with a virgin PTFE material.

FIG. 6A is a chart with results showing a cell cycle analysis by propidium iodide of HeLa cells in wells coated with a spray PTFE material.

FIG. 6B is a chart with results showing a cell cycle analysis by propidium iodide of HeLa cells in wells coated with a modified PTFE material.

FIG. 6C is a chart with results showing a cell cycle analysis by propidium iodide of HeLa cells in wells coated with a virgin PTFE material.

FIG. 7A is a chart with results showing a cell cycle analysis by propidium iodide of 3T3 cells in wells coated with a spray PTFE material.

FIG. 7B is a chart with results showing a cell cycle analysis by propidium iodide of 3T3 cells in wells coated with a modified PTFE material.

FIG. 7C is a chart with results showing a cell cycle analysis by propidium iodide of 3T3 cells in wells coated with a virgin PTFE material.

FIG. 8 is a table with results showing growth of MDA-MB-231 cells from PTFE-coated wells over a period of 120 hours.

FIG. 9 is a table with results showing compared growth of HeLa and 3T3 cells after incubation and plating with fresh media.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.” Where a range of values is disclosed, the respective embodiments includes each value between the upper and lower limits of the range.

Generally, as described in more detail below, a cultureware system includes a coating of polytetrafluoroethylene (“PTFE”) material (also referred to as TEFLON®) that renders the cultureware (i) reusable for long periods of time (e.g., up to months of repeated use), (ii) amenable to long-term (e.g., up to 5 days) maintenance of cells with adherent phenotypes, but without allowing the cells to adhere to container surfaces, (iii) able to preserve cells in a cytostatic, yet metabolically-active, state, and (iv) allow quantitative recovery of cells. The coating of PTFE material, which impedes cell adhesion, provides these and other benefits for a reusable cultureware system.

To circumvent issues associated with conventional cultureware and cell dissociation reagents, holding containers for cells have been prepared from a plastic (e.g., polystyrene) 12-well plates lined with PTFE. In other embodiments, the holding containers for cells are prepared from polystyrene 6-well plates or polystyrene 24-well plates. Such wells promote the ability to “hold cells” for use without requiring reagents to subsequently remove cells from plates. Moreover, because Materials sold as “TEFLON®” often differ in chemical composition among manufacturers, and to ensure that results are generally representative of a variety of commercially available products, three different PTFE products were tested in the fabrication of the described cultureware.

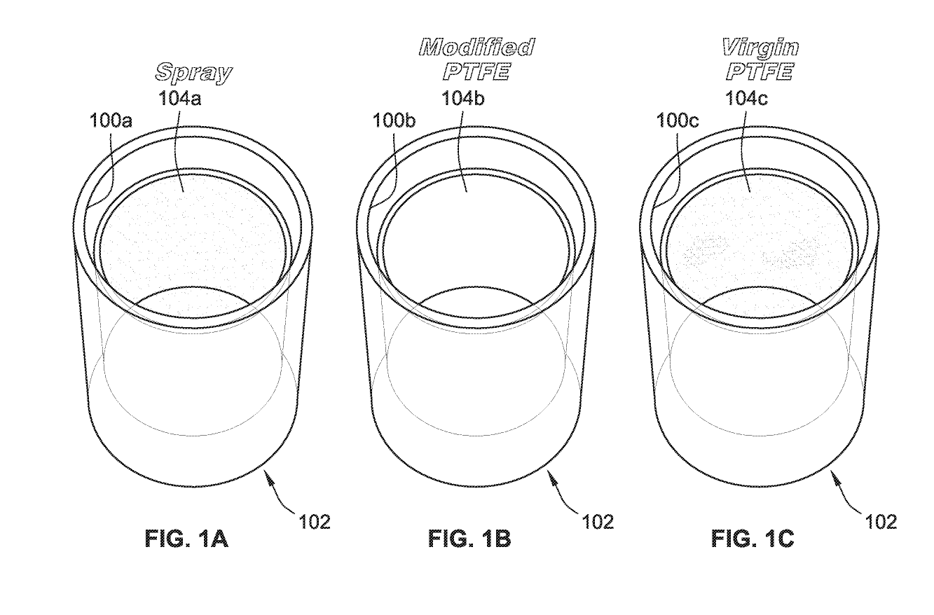

Referring to FIGS. 1A-1C, individual wells 100a-100c of a twelve-well polystyrene plate 102 are coated, respectively, with a spray TEFLON® coating 104a, a modified PTFE coating 104b, and a virgin PTFE coating 104c. Specifically, in reference to FIG. 1A, a first well 100a is coated with a dry-film lubricant TEFLON® spray 104a, which consists of TEFLON® coated ceramic particles. In reference to FIG. 1B, a second well 100b is lined with PTFE 104b that is in a modified form having a backing for use with glues and adhesives (also referred to as modified PTFE 104b). In reference to FIG. 1C, a third well 100c is lined with cut and adhesive-backed virgin PTFE sheets 104c.

For lined plates, sheets of modified and virgin PTFE are backed with double-sided adhesive and, then, cut into the circular and rectangular shapes designed to fit and line the bottoms and sides of wells in a 12-well plate, for example. Plates are more easily and quickly coated with the TEFLON® spray 104a. Because the spray does not require adhesive to adhere to the well 100a-100c, this approach eliminates the possibility of cells leaking through a junction created by two adjacent sheets and adhering to the polystyrene plate.

A comparison study between (a) results from monitoring several metrics of population health and (b) results from cultures maintained on traditional polystyrene cultureware (c) shows the effectiveness of the non-stick PTFE cultureware of the present description. The treated wells 100a-100c were seeded with HeLa, MDA-MB-231, and 3T3 cells. The measurements included: (i) cell recovery from the wells 100a-100c, (ii) cell viability, and (iii) cell cycle stage once per day for a total of five days after seeding. Additionally, cells were seeded after being recovered from the coated wells 100a-100c at each time point in traditional polystyrene well plates and the respective cell growth was examined over the course of three days. Additionally, cells for each five day experiment were seeded from the same culture to more effectively compare results between samples.

In the study, materials were characterized to determine hydrophobicity using contact angle measurements of water. Additionally, scanning electron microscope (SEM) images of sprayed plates were taken to determine how the TEFLON® spray coats a plate surface and, therefore, how particles may interact with cells. Because dissociation reagents are not required for cell collection in PTFE-treated wells, cells can be removed gently by pipetting.

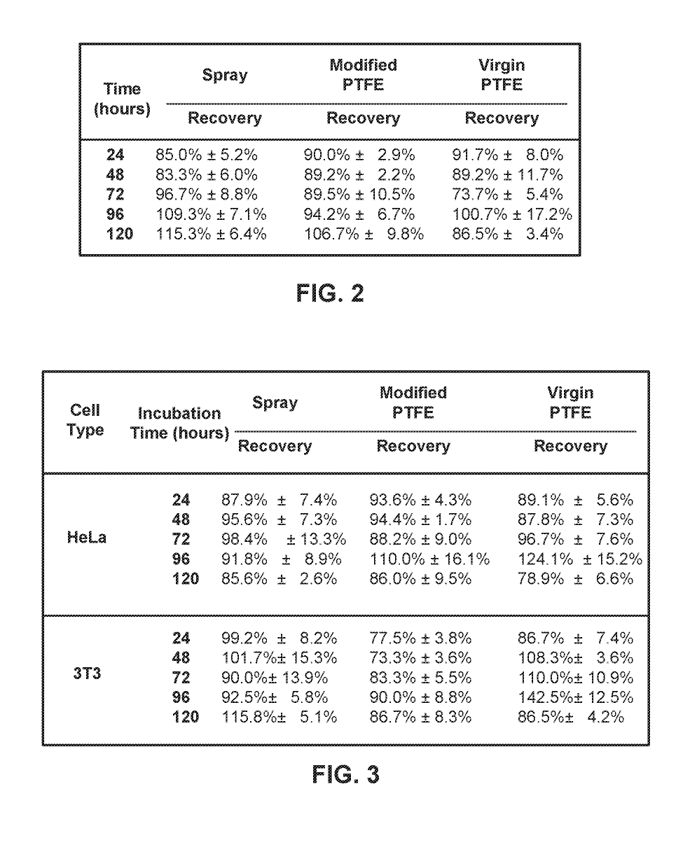

Referring to FIGS. 2 and 3, the study determined that ˜90% of cells seeded in PTFE-treated wells can be recovered. According to some examples, the recovery of mammalian cells, within about 120 hours from inserting a culture of adherent mammalian cells into a cell receiver of a disposable holding container, is in the range of about 85% to about 100%. In FIG. 2, results show recovery of MDA-MB-231 cells from TEFLON®-coated wells seeded with 1×10⁵ cells over a period of 120 hours (n=3 replicates). In FIG. 3, results show recovery HeLa and 3T3 cells from TEFLON®-coated wells over a period of 120 hours (n=3 replicates). The study indicates that the cells are either not adhering to the “non-stick” plates, or that the adhesion forces are very weak and are potentially reversed by small forces introduced by pipetting. Accordingly, any cell loss is likely due to pipetting error or loss of cells in the junctions where two pieces of PTFE interact when using the plates lined with PTFE sheets. The recovery data further shows that little to no growth is occurring in the cell population during the incubation in these PTFE wells for up to five days.

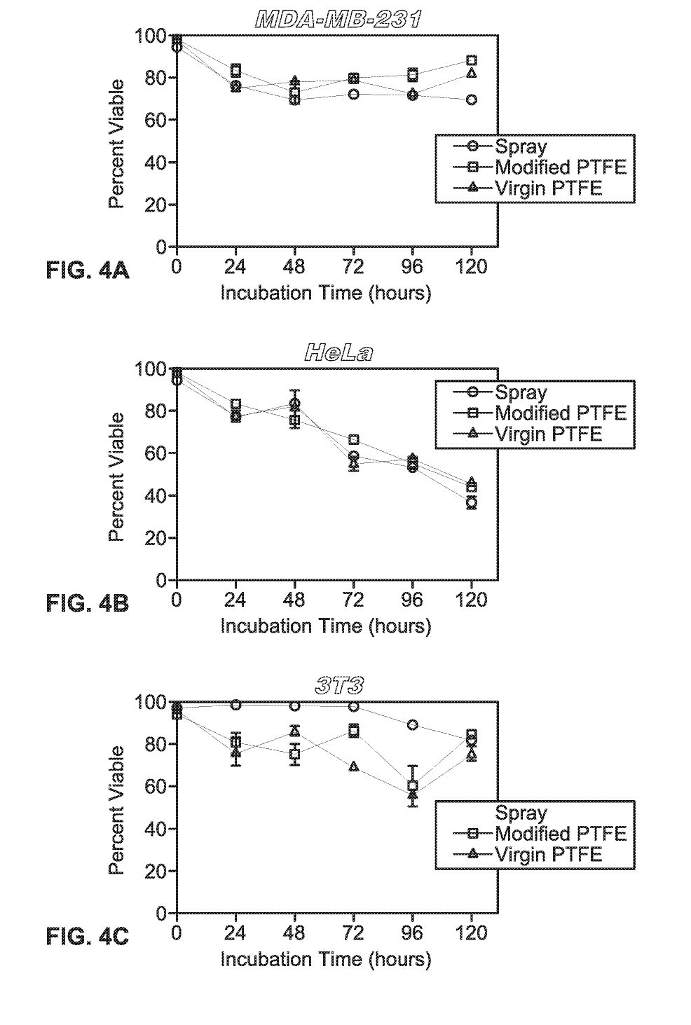

Referring to FIG. 4A-4C, the viability of the cells was measured with propidium iodide to determine if these adherent cell lines were capable of surviving without normal adhesion to the culture surface to stimulate their normal growth phenotype. Specifically, cell viability of MDA-MB-231 cells (FIG. 4A), HeLa cells (FIG. 4B), and 3T3 cells (FIG. 4C) were incubated in culture wells lined with TEFLON® spray, modified PTFE, and virgin PTFE as measured by propidium iodide staining over a period of 120 hours. In reference to FIG. 4A, MDA-MB-231 cells remained the most viable over the period of 120 hours for all three well types. Viability dropped within the first 24 hours, but was maintained around 80% over the next 96 hourss.

3T3 cells displayed a more significant drop in viability over 120 hours (to ˜60%) when incubated in plates lined with PTFE sheets. Referring to FIG. 4C, however, cells incubated in plates coated with the TEFLON® spray remained over 90% viable for the first 72 hours and only decreased slightly over the next 48 hours. Thus, according to some examples, a decreased cell adhesion results in a cell viability rate of at least 60%, at least 70%, and, preferably, at least 90% or more over at least a 72-hour culturing period. Referring to FIG. 4B, HeLa cells demonstrated a steady decrease in viability over time across all three materials. The steady level of viable cells throughout the study shows that if cells are adhering to PTFE, the forces are very weak as cells are not shearing when removed.

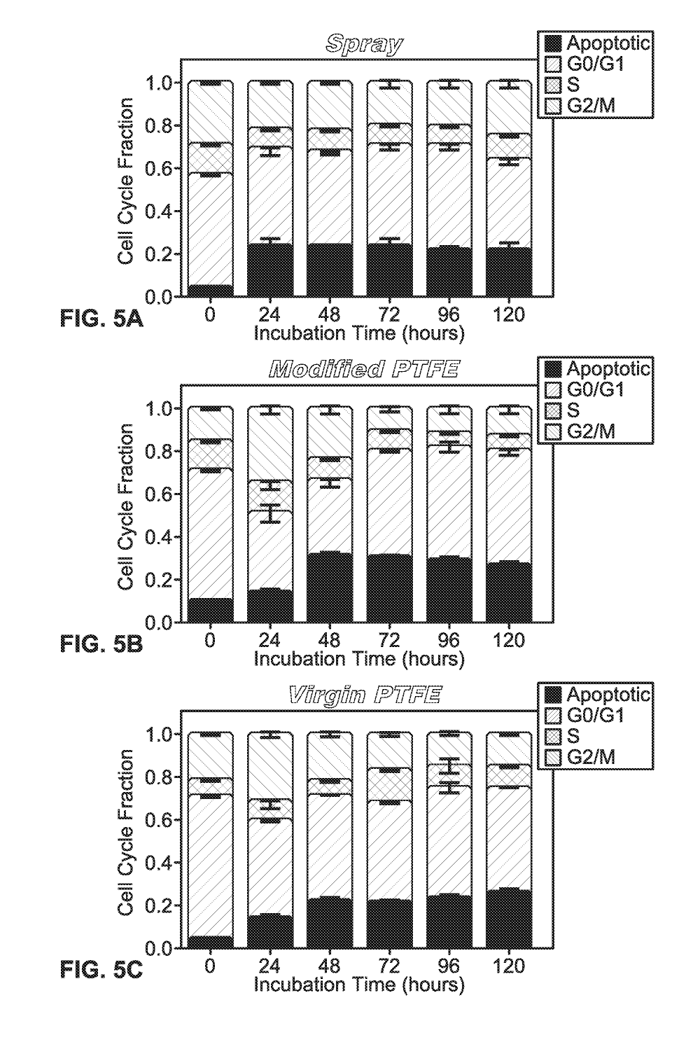

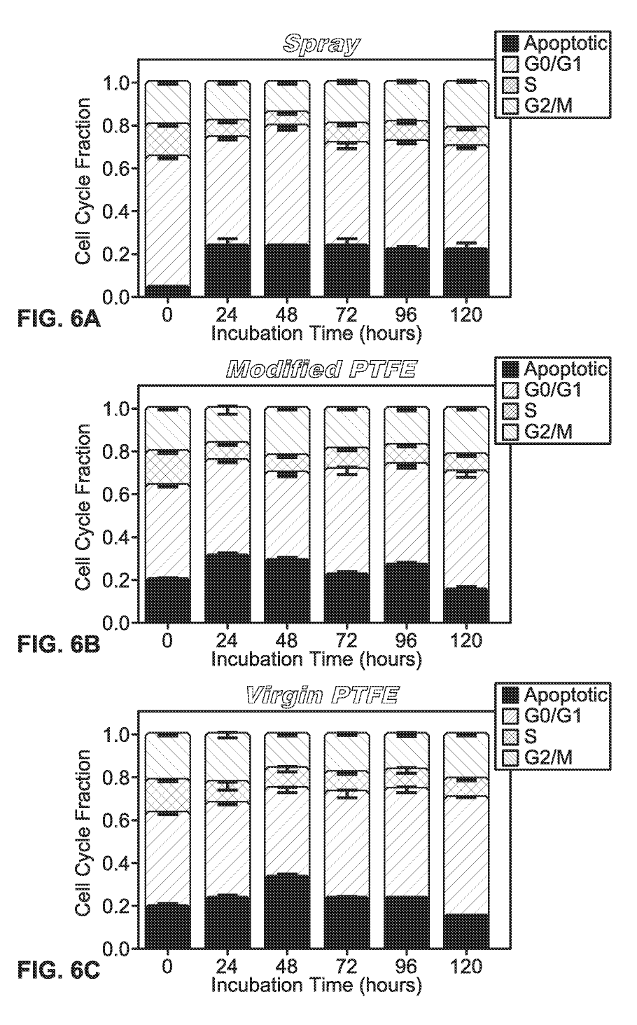

Referring generally to FIGS. 5A-5C, 6A-6C, and 7A-7C, based on the retention of viable cells and no substantial increase in recovery of cell numbers, the effect of incubating cells in a ‘non-stick’ environment on cellular metabolism was further explored. Initially, the study reviewed cell cycle stages. Apart from an increased number of apoptotic cells, MDA-MB-231 cells appeared to have very little change in their cell cycle population as a whole when incubated in sprayed wells over the 120 hour period.

Referring specifically to FIGS. 5A-5C, cell cycle analysis by propidium iodide of MDA-MB-231 cells were incubated over a period of 120 hours in wells coated with TEFLON® spray (FIG. 5A), modified PTFE (FIG. 5B), and virgin PTFE (FIG. 5C) (N>20000 events per measurement, n=3 replicates). In lined wells, cells experienced an increased percentage in either mitosis or G2 within the first 24 hours, but in the last 72 hours, returned to values comparable to those seen in sprayed wells.

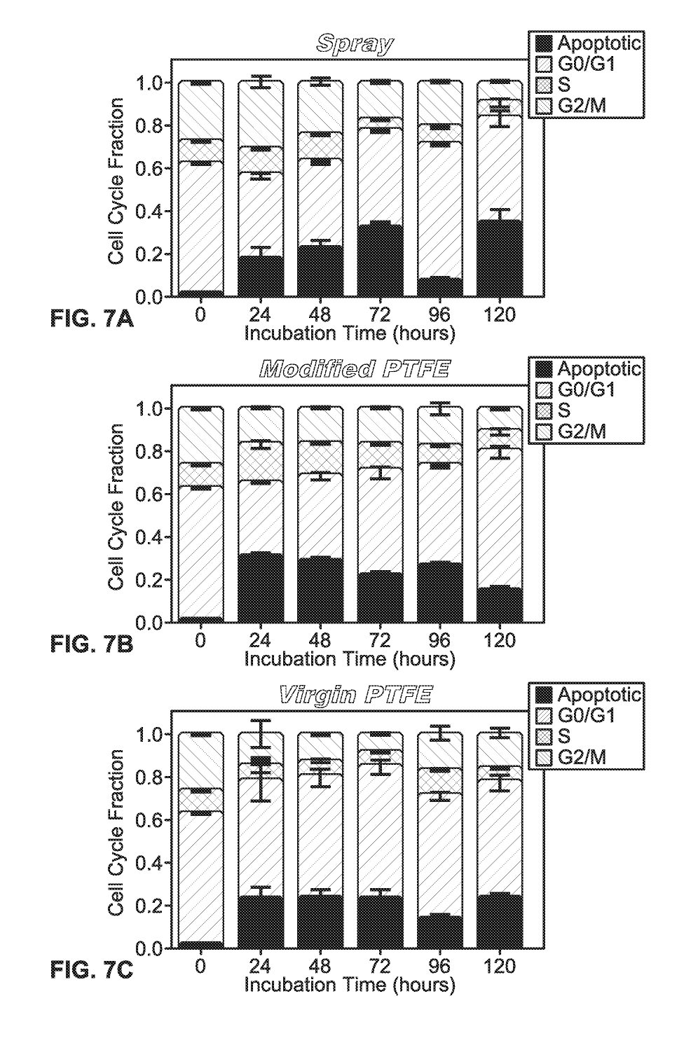

Referring specifically to FIGS. 6A-6C cell cycle analysis by propidium iodide of HeLa cells were incubated in TEFLON® spray (FIG. 6A), modified PTFE (FIG. 6B), and virgin PTFE (FIG. 6C) lined wells over a period of 120 hours. Referring specifically to FIGS. 7A-7C cell cycle analysis by propidium iodide of 3T3 cells were incubated in TEFLON® spray (FIG. 6A), modified PTFE (FIG. 6B), and virgin PTFE (FIG. 6C) lined wells over a period of 120 hours. The results show similar phenomena observed in 3T3 and HeLa cells across all three material types. This data coupled with the cell recovery data shows that cells may enter a temporary state of cytostasis while incubated in the wells.

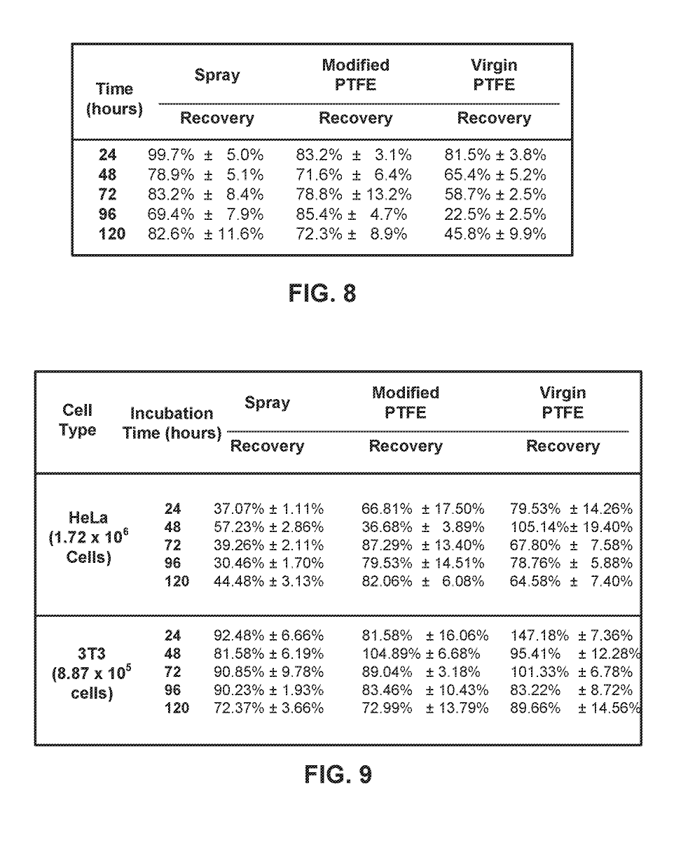

Referring generally to FIGS. 8 and 6A-6C, further results demonstrate the use of PTFE wells as temporary holding containers for viable cells. In the respective experiments, incubated cells were plated and growth was compared to cells cultured on polystyrene dishes. MDA-MB-231 cells held in TEFLON® spray and modified PTFE wells grew to a population that averaged ˜80% of the control samples.

Referring specifically to FIG. 8, in virgin PTFE wells, however, recovered cells were unable to grow at normal rates. The results show growth of MDA-MB-231 cells from TEFLON®-coated wells over a period of 120 hours (N>20000 events per measurement, n=3 replicates). Grown cells that were not incubated in wells totaled 4.93×10⁵ cells in 6-well plates. These results are likely based on differences in chemical composition and topography among the materials, where different interactions may affect general cell function.

Referring specifically to FIGS. 6A-6C, similar results were found with HeLa cells, in which growth was slightly hindered when cells were incubated in wells lined with PTFE, but were unable to return to a normal growth pattern when incubated in TEFLON® spray wells. 3T3 cells, however, were only slightly affected by incubation in any of the well types and were capable of growing at rates similar to those of the control, indicating that cell type may also contribute to behavior in PTFE coated wells.

### Materials and Methods

In reference to Preparation and Handling of PTFE-coated wells, sheets of PTFE (virgin PTFE from ePlastics and PTFE with an adhesive-ready backing purchased from McMaster-Carr) were backed with a double-sided permanent adhesive (Flexcon). Sheets were cut into dimensions of 12-well plates, with circular inserts that were 22 millimeters (“mm”) in diameter and that were cut to line the bottom of each well. Furthermore, strips of 69 mm×8 mm were cut to line the sides of each well to minimize the contact of cells with the supporting polystyrene surface of the plate. 12-well plates were further coated with TEFLON® non-stick dry-film lubricant. One layer of particles was sprayed across the surface and allowed to dry in air before applying two additional layers to ensure complete coverage of the plastic. The wells were (1) sterilized by rinsing with 20% bleach, then (2) rinsed twice with PBS, and (3) incubated in PBS at 37° C. for 30 minutes to remove any residual bleach. Plates were, then, placed under UV irradiation in laminar flow hoods and were immediately useable. When plates were reused, the same cleaning protocol was utilized.

In reference to Cell Culture, HeLa, MDA-MB-231, and 3T3 cells (ATCC) were cultured in Dulbecco's Modified Eagle Medium (DMEM; Corning) supplemented with 10% Fetal Bovine Serum (FBS; Biowest) at 37° C. and 5% CO₂. MCF-7 cells (ATCC) were cultured in Dulbecco's Modified Eagle's Medium (DMEM; Corning) and 10% FBS at 37° C. and 5% CO₂. Cells were grown in T-175 flasks (Falcon) to 80% confluency and collected from the dish using 0.43 mM EDTA. Cell density was adjusted to 10,000 cells/milliliter (“mL”) and 1 mL of the final cell solution was deposited in each well with an additional 0.5 mL of fresh media, resulting in a total volume of 1.5 mL. For time point measurements, all cells came from the same culture, and could therefore be compared to each other and a t=0 measurement, taken from the initial stock of cells. When in wells, cells were allowed to incubate in their original media (DMEM, 10% FBS) without subsequent exchange for the entirety of their designated time point at 37° C. and 5% CO₂.

Referring to FIGS. 8 and 9, growth was tested after incubation in wells. Generally, cells were collected, washed once in fresh media, and plated in 6-well plates. Then, the cells were allowed to grow for 72 hours before counting. The number of the cells was compared to growth of cells that were not incubated in TEFLON®-treated wells.

In reference to Cell Counting, cells were collected from TEFLON®-treated wells by pipette and dispensed into cylindrical tubes. Wells were washed twice with PBS to collect any remaining cells. Cells were sedimented by centrifugation at 200 g for 5 minutes. The medium was aspirated and the cell pellet was washed once with PBS. After the final wash, the cell pellet was resuspended in PBS, and cells were counted using a hemocytometer or a COUNTESS® II Automated Cell Counter (ThermoFisher). To count samples plated to test growth, cells were treated with 0.43 mM EDTA, collected, washed once with PBS, and counted on a hemocytometer or a COUNTESS® II Automated Cell Counter.

In reference to Cell Viability Analysis, cells were collected from the wells and plates were washed twice with PBS to collect any remaining cells. Specifically, after incubation in TEFLON®-coated wells, HeLa and 3T3 cells were plated with fresh media in 6-well plates and allowed to grow for 72 hours. Cell counts were collected and then centrifuged, washed once with PBS, and resuspended in 200 microliters (“μL”) of PBS. 2 μL of 50 μg/mL propidium iodide (Biotium) was added to each sample, which was immediately analyzed by flow cytometry (Guava easyCyte 6HT-2L) in the Yellow (583/26 nm) log channel. Gain was adjusted using unstained cells to shift autofluorescence signal below 10¹ and any signal above was considered positive, indicating cell death.

In reference to Cell Cycle Analysis, recovered cells were fixed and permeabilized with ice cold 70% ethanol, which was added dropwise while vortexing on a low setting to create a single cell suspension. Cells were then incubated at 4° C. for 1 hour, washed once with PBS, resuspended in 50 μL of 100 μg/mL RNase A (Amresco) and 200 μL of 50 μg/mL propidium iodide. Samples were then incubated at room temperature for 30 minutes before being analyzed by flow cytometry in the Yellow (583/26 nm) linear channel.

Each of these embodiments and obvious variations thereof is contemplated as falling within the spirit and scope of the claimed invention, which is set forth in the following claims. Moreover, the present concepts expressly include any and all combinations and sub-combinations of the preceding elements and aspects.

What is claimed is:

1. A cultureware system for maintaining viability of mammalian cells, the cultureware system comprising:
a disposable holding container having a cell receiver with a cell-receiving surface, the cell receiver consisting of a cell-adhesion inducement material including at least one of a plastic material and a glass material;
a polytetrafluoroethylene (PTFE) coating lining the cell-adhesion inducement material of the cell-receiving surface; and
a culture of adherent mammalian cells located within the cell receiver on the PTFE coating;
wherein, in response to attachment interaction between the mammalian cells and the PTFE coating, a decreased cell adhesion results in a cell viability rate in the range of at least about 60% to about 70% over at least a 72-hour culturing period, the cell viability rate being under 90% over the at least 72-hour culturing period if, in the absence of the PTFE coating, the mammalian cells are located directly on the cell-adhesion inducement material.

2. The cultureware system of claim 1, wherein a range of about 85% to about 100% of the mammalian cells are recoverable within 120 hours from inserting the culture of adherent mammalian cells into the cell receiver of the disposable holding container.

3. The cultureware system of claim 1, wherein the PTFE coating renders the disposable holding container reusable with other subsequent cultures of adherent mammalian cells.

4. The cultureware system of claim 1, wherein the PTFE coating renders the disposable holding container reusable for at least two months of repeated use after an initial use.

5. The cultureware system of claim 1, wherein the mammalian cells are preserved in a cytostatic, metabolically active state.

6. The cultureware system of claim 1, wherein the disposable holding container is a 6-well polystyrene plate, a 12-well polystyrene plate, or a 24-well polystyrene plate.

7. The cultureware system of claim 1, wherein the PTFE coating is selected from a group consisting of (a) a dry-film lubricant spray with PTFE-coated ceramic particles, (b) a modified PTFE coating with an adhesive backing, and (c) a virgin PTFE coating with an adhesive backing.

8. A cell-culture holding container for cultured mammalian cells, the holding container comprising:
a receiver plate with a plurality of receiver wells;
a plurality of receiver inserts positioned, respectively, in the plurality of receiver wells, each receiver insert of the plurality of receiver inserts having an internal receiver surface;
a polytetrafluoroethylene (PTFE) coating applied to each internal receiver surface; and
a culture of adherent mammalian cells located within the plurality of receiver inserts, the PTFE coating being interposed between the mammalian cells and the respective internal receiver surface to impede cell adhesion to the internal receiver surface.

9. The cell-culture holding container of claim 8, wherein a range of about 85% to about 100% of the mammalian cells are recoverable within 120 hours from inserting the culture of adherent mammalian cells into the plurality of receiver inserts of the receiver plate.

10. The cell-culture holding container of claim 8, wherein the PTFE coating renders the receiver plate reusable with other subsequent cultures of adherent mammalian cells.

11. The cell-culture holding container of claim 8, wherein the PTFE coating renders the receiver plate reusable for at least two months of repeated use after an initial use.

12. The cell-culture holding container of claim 8, wherein the mammalian cells are preserved in a cytostatic, metabolically active state.

13. The cell-culture holding container of claim 8, wherein the receiver plate is a 6-well polystyrene plate, a 12-well polystyrene plate, or a 24-well polystyrene plate.

14. The cell-culture holding container of claim 8, wherein the PTFE coating is selected from a group consisting of (a) a dry-film lubricant spray with PTFE-coated ceramic particles, (b) a modified PTFE coating with an adhesive backing, and (c) a virgin PTFE coating with an adhesive backing.

15. A method of culturing mammalian cells in a holding container, the method comprising:
providing a receiver plate with a plurality of receiver wells;
inserting a plurality of receiver inserts into respective ones of the plurality of receiver wells;
coating an internal receiver surface of each of the plurality of receiver wells with a polytetrafluoroethylene (PTFE) material to achieve a PTFE-coated receiver surface;
culturing adherent mammalian cells on the PTFE-coated receiver surface; and
in response to the culturing of the mammalian cells on the PTFE-coated receiver surface, achieving a cell viability rate of at least 70% over at least a 72-hour culturing period.

16. The method of claim 15, further comprising recovering a range of about 85% to about 100% of the mammalian cells within 120 hours from initiating the culturing.

17. The method of claim 15, further comprising rendering, in response to the coating with the PTFE material, the receiver plate reusable with other subsequent cultures of adherent mammalian cells.

18. The method of claim 15, further comprising rendering, in response to the coating with the PTFE material, the receiver plate reusable for at least two months after an initial use.

19. The method of claim 15, further comprising, in response to the coating with the PTFE material, preserving the mammalian cells in a cytostatic, metabolically active state.

20. The method of claim 15, wherein the PTFE material is selected from a group consisting of (a) a dry-film lubricant spray with PTFE-coated ceramic particles, (b) a modified PTFE coating with an adhesive backing, and (c) a virgin PTFE coating with an adhesive backing.
